(19) United States
(12) Patent Application Publication
(10) Pub. No.: US 2007/0020214 A1
(43) Pub. Date: Jan. 25, 2007

(54) Dual or multiple phase cosmetic agent with improved reversible mixing and separation behavior

(76) Inventors: Burkhard Muller, Hamburg (DE); Thorsten Knappe, Schenefeld (DE); Birgit Rautenberg-Groth, Ellerau (DE)

Correspondence Address: PAUL & PAUL, 2900 TWO THOUSAND MARKET STREET, PHILADELPHIA, PA, 19103, US

(21) Appl. No.: 11/471,103

(22) Filed: Jun. 19, 2006

Related U.S. Application Data
(63) Continuation of application No. PCT/EP04/12906, filed on Nov. 13, 2004.

(30) Foreign Application Priority Data
Dec. 19, 2003 (DE) 103 60 688.2

(51) Int. Cl. A61K 8/37

(52) U.S. Cl. 424/70.2; 424/70.7

(57) ABSTRACT

The invention relates to a method for the permanent deformation of keratin fibers. According to said method, the fibers are treated with an aqueous preparation of a keratin-reducing substance prior to and/or after a mechanical deformation, then after a certain exposure time, the fibers are rinsed again with a first rinsing agent, fixed with an aqueous preparation of an oxidant, rinsed again after an exposure time and optionally treated again. Said method is particularly effective and kind to the fibers. The mixing and separation behavior is improved if at least one of the two aqueous preparations or the first rinsing agent is in the form of a multiple phase system containing at least one oil component and at least one N-acyl glutamic acid or the salts thereof. The multiple phase system is converted into a homogeneous system for a given time period by a mechanical movement for application to the fibers and separates again during the re-formation of the multiple phase system.

### CROSS-REFERENCE TO RELATED APPLICATIONS

This application is a continuation under 35 U.S.C. §365(c) and 35 U.S.C. §120 of International Application PCT/EP2004/012906, filed Nov. 13, 2004. This application also claims German priority under 35 U.S.C. §119 of 103 60 688.2, filed Dec. 19, 2003.

### STATEMENT REGARDING FEDERALLY SPONSORED RESEARCH OR DEVELOPMENT

Not Applicable

### INCORPORATION-BY-REFERENCE OF MATERIAL SUBMITTED ON A COMPACT DISC

Not Applicable

### BACKGROUND OF THE INVENTION

(1) Field of the Invention

The invention relates to a method for the permanent deformation of keratin fibers, in particular, of human hair, by reductive cleavage and oxidative relinking of disulfide bonds of the keratin, and to multiple phase agents with improved mixing and separation behavior suitable for this method.

The permanent deformation of keratin fibers is usually carried out by mechanically deforming the fibers and securing the deformation by suitable aids. Before and/or after this deformation, the fibers are treated with the aqueous preparation of a keratin-reducing substance and, after a contact time, rinsed with water or an aqueous solution. In a second step, the fibers are then treated with the aqueous preparation of an oxidizing agent. After a contact time, this too is rinsed out and the fibers are freed from mechanical deformation aids (rollers, papillotes).

The aqueous preparation of the keratin-reducing agent has usually been rendered alkaline so that first an adequate fraction of the thiol functions is present in deprotonated form and second the fiber swells and in so doing enables the keratin-reducing substance to penetrate deeply into the fiber. The keratin-reducing substance cleaves some of the disulfide bonds of the keratin to give —SH groups, resulting in a loosening of the peptide crosslinking and consequently the tensioning of the fiber as a result of the mechanical deformation to give a new orientation of the keratin structure. Under the influence of the oxidizing agent, disulfide bonds are relinked, and in so doing the keratin structure is newly fixed in the pregiven deformation. A known method of this type is the permanent waving treatment of human hair. This can be used either for producing curls and waves in straight hair and for straightening curled hair.

However, one negative consequence of the permanent waving of hair carried out in this way is often that the hair becomes brittle and dull. In addition, in many cases, other properties, such as wet and dry combability, feel, suppleness, softness, sheen and tear resistance are adversely affected.

(2) Description of Related Art, Including Information Disclosed Under 37 C.F.R. §§ 1.97 and 1.98.

In the past there has therefore been no lack of attempts to overcome this.

Appropriate modification of the reducing solution generally does not lead to satisfactory wave results. The addition of known additives such as structurants, polymers, film formers and crosslinking resins, or the neutral to slightly acidic adjustment of the preparation can reduce hair damage, but the hair structure remains weakened to a greater or lesser degree. Although care treatment of the hair through further after-treatments can again improve the hair properties, it does require additional time expenditure and usually the use of at least one other agent.

In addition, it was therefore the object to find a method for the permanent deformation of keratin fibers in which the specified undesired side-effects are further reduced or excluded altogether.

(2) Description of Related Art, Including Information Disclosed Under 37 C.F.R. §§ 1.97 and 1.98.

It has already been disclosed, in the patent application WO-A1-99/58099, that a significant improvement in the properties of deformed keratin fibers, such as improved combability and vitality, is achieved by at least one agent used during the deformation method being formulated as dual or multiple phases and comprising specific compounds. In addition, the laid-open specification W0-A2-02/67881 discloses that the combability and vitality can be further improved through the use of silicone oils with selected kinematic viscosity in such dual or multiple phase agents.

To realize a constantly effective action of a dual or multiple phase agent, on the one hand, the homogeneous mixing of the phases prior to application, and on the other hand, the reversibility of this mixture is decisive. However, in the multiple phase agents of the prior art, the mixing and separation behavior of the phases following application is not satisfactory. Especially in cases of repeated mixing and separation, complete mixing and, in particular, separation of the phases is not satisfactorily ensured.

### BRIEF SUMMARY OF THE INVENTION

Therefore, the invention provides a method for the permanent deformation of keratin fibers in which the fibers are treated before and/or after a mechanical deformation with an aqueous preparation of a keratin-reducing substance, after a contact time, rinsed with a first rinse, then neutralized with an aqueous preparation of an oxidizing agent and, likewise after a contact time, rinsed and optionally after-treated, characterized in that at least one of the two aqueous preparations or the first rinse is in the form of a dual or multiple phase system which comprises at least one oil component and, as emulsifier, at least one N-acylglutamic acid or salts thereof, and which is reversibly converted into a homogeneous system for application to the fibers through mechanical movement.

In the text below, the following names are used:
“waving agent” for the aqueous preparation of the keratin-reducing substance,
“intermediate rinse” for the first rinse and
“neutralizer” for the aqueous preparation of the oxidizing agent.

### BRIEF DESCRIPTION OF THE SEVERAL VIEWS OF THE DRAWING(S)

Not Applicable

### DETAILED DESCRIPTION OF THE INVENTION

In the method according to the invention, the waving agent, the intermediate rinse and/or the neutralizer are formulated in the form of a dual or multiple phase system. Dual and multiple phase systems used according to the invention are systems in which at least two separate continuous phases are present. Examples of such systems are preparations which have the following phases:
an aqueous phase and a nonaqueous phase, which are present separately from one another
an aqueous phase and two nonaqueous phases which are immiscible with one another, and which are each present separately
an oil-in-water emulsion and, separately from this, a nonaqueous phase
a water-in-oil emulsion and, separately from this, an aqueous phase.

No dual phase systems for the purposes of the present invention are systems in which there is only one continuous phase like, for example, pure oil-in-water or water-in-oil emulsions.

The agents having dual or multiple phase systems and used in the method according to the invention only develop their full effect, however, when they are applied to the keratin fibers in homogeneous form. For this, the agents are reversibly converted into homogeneous systems through mechanical influence, e.g., simple shaking of the container containing them by hand. In order to ensure homogeneous application to the keratin fibers, this homogeneous state must be retained for an adequate time before the individual phases form again. For the teaching according to the invention, it has proven sufficient if this homogeneous state is stable for at least 20 seconds, in particular, at least 30 seconds, before an interfacial layer and thus the formation of the individual phases is again recognizable for the viewer. After a certain period, preferably of at most 60 minutes, particularly preferably of at most 30 minutes, the reversible mixing of the phases by separation is complete, so that the starting state with preservation of the dual or multiple phase system is achieved again.

In addition to water, the dual and multiple phase systems used according to the invention obligatorily comprise at least one oil component.

Oil components suitable according to the invention are in principle all oils and fatty substances, and mixtures thereof with solid paraffins and waxes. Preference is given to those oil components whose solubility in water at 20° C. is less than 1% by weight, in particular, less than 0.1% by weight. The melting point of the individual oil or fat components is preferably below about 40° C. Oil components which are liquid at room temperature, i.e. below 25° C., may be particularly preferred according to the invention. When using a plurality of oil and fat components and optionally solid paraffins and waxes, however, it is usually also sufficient if the mixture of the oil and fat components and optionally paraffins and waxes satisfies these conditions.

The oil components are preferably present in the agents of the method according to the invention in the weight ratio range to the remaining components of from 1:200 to 1:1, particularly preferably from 1:40 to 1:1, very particularly preferably from 1:20 to 1:3.

A preferred group of oil components are vegetable oils. Examples of such oils are apricot kernel oil, avocado oil, sunflower oil, olive oil, soy oil, rapeseed oil, almond oil, jojoba oil, orange oil, wheat germ oil, peach kernel oil and the liquid fractions of coconut oil. Also suitable, however, are other triglyceride oils, such as the liquid fractions of beef tallow, and synthetic triglyceride oils.

A further particularly preferred group of oil components which can be used according to the invention are liquid paraffin oils and synthetic hydrocarbons, and di-n-alkyl ethers with in total between 12 and 36 carbon atoms, in particular, 12 to 24 carbon atoms, such as, for example, di-n-octyl ether, di-n-decyl ether, di-n-nonyl ether, di-n-undecyl ether, di-n-dodecyl ether, n-hexyl n-octyl ether, n-octyl n-decyl ether, n-decyl n-undecyl ether, n-undecyl n-dodecyl ether and n-hexyl n-undecyl ether, and di-tert-butyl ether, diisopentyl ether, di-3-ethyldecyl ether, tert-butyl n-octyl ether, isopentyl n-octyl ether and 2-methylpentyl n-octyl ether. The commercially available compounds 1,3-di(2-ethylhexyl)cyclohexane (Cetiol® S) and di-n-octyl ether (Cetiol® OE) may be preferred.

Oil components which can likewise be used according to the invention are fatty acid and fatty alcohol esters. Preference is given to the monoesters of fatty acids with alcohols having 3 to 24 carbon atoms. This group of substances are [products of the esterification of fatty acids having 8 to 24 carbon atoms, such as, for example, capronic acid, caprylic acid, 2-ethylhexanoic acid, capric acid, lauric acid, isotridecanoic acid, myristic acid, palmitic acid, palmoleic acid, stearic acid, isostearic acid, oleic acid, elaidic acid, petroselic acid, linoleic acid, linolenic acid, elaeostearic acid, arachidic acid, gadoleic acid, behenic acid and erucacic acid, and technical-grade mixtures thereof, which are produced, for example, during the pressurized cleavage of natural fats and oils, during the reduction of aldehydes from the Roelen oxo synthesis or the dimerization of unsaturated fatty acids, with alcohols, such as, for example, isopropyl alcohol, glycerol, caproic alcohol, capryl alcohol, 2-ethylhexyl alcohol, capric alcohol, lauryl alcohol, isotridecyl alcohol, myristyl alcohol, cetyl alcohol, palmoleyl alcohol, stearyl alcohol, isostearyl alcohol, oleyl alcohol, elaidyl alcohol, petroselinyl alcohol, linolyl alcohol, linolenyl alcohol, elaeostearyl alcohol, arachyl alcohol, gadoleyl alcohol, behenyl alcohol, erucyl alcohol and brassidyl alcohol, and technical-grade mixtures thereof which are produced, for example, during the high-pressure hydrogenation of technical-grade methyl esters based on fats and oils or aldehydes from the Roelen oxo synthesis, and as monomer fraction during the dimerization of unsaturated fatty alcohols. Of particular preference according to the invention are isopropyl myristate, isononanoic acid C16-18-alkyl esters (Cetiol® SN), 2-ethylhexyl stearate (Cetiol® 868), cetyl oleate, glycerol tricaprylate, coconut fatty alcohol caprate/caprylate and n-butyl stearate.

In addition, dicarboxylic acid esters, such as di-n-butyl adipate, di(2-ethylhexyl) adipate, di(2-ethylhexyl) succinate and diisotridecyl acetate, and also diol esters, such as ethylene glycol dioleate, ethylene glycol diisotridecanoate, propylene glycol di(2-ethylhexanoate), propylene glycol diisostearate, propylene glycol dipelargonate, butanediol diisostearate and neopentyl glycol dicaprylate also represent oil components which can be used according to the invention, as do complex esters, such as, for example, diacetyl glycerol monostearate.

Finally, hydrophobic components which can preferably be used according to the invention are also silicone oils, in particular, dialkyl- and alkylarylsiloxanes, such as, for example, dimethylpolysiloxane and methylphenylpolysiloxane, and alkoxylated and quaternized analogs thereof, and cyclic siloxanes. Examples of such silicones are the products sold by Dow Corning under the names DC 190, DC 200 and DC 1401, and the commercial products DC 344 and DC 345 from Dow Corning, Q2-7224 (manufacturer: Dow Corning; a stabilized trimethylsilylamodimethicone), Dow Corning® 929 emulsion (comprising a hydroxylamino-modified silicone, which is also referred to as amodimethicone), SM-2059 (manufacturer: General Electric), SLM-55067 (manufacturer: Wacker), and Abil®-Quat 3270 and 3272 (manufacturer: Th. Goldschmidt; diquaternary polydimethylsiloxanes, Quaternium-80). For the purposes of the invention, preference may be given to silicone oils with a kinematic viscosity up to 50,000 cSt measured at 25° C. Very particular preference is given to silicone oils with kinematic viscosities up to 10,000 cSt measured at 25° C. The viscosities are determined here in accordance with the falling sphere method corresponding to the British Standard 188 method. Comparable results are obtained using manufacturers' test procedures analogous to British Standard 188, for example, the “CTM 0577” from Dow Corning Corporation.

In a particular embodiment, the hydrophobic components used are, in particular, cyclic siloxanes, such as, for example, the products Dow Corning® 344, Dow Corning® 345, Dow Corning® 244, Dow Corning® 245 or Dow Corning® 246 with kinematic viscosities of up to 10,000 cSt at 25° C. determined in accordance with the manufacturer's instructions.

The N-acylglutamic acid and salts thereof obligatorily present as emulsifier in the dual or multiple phase agents of the method according to the invention is particularly preferably used in combination with a silicone oil as oil component in the dual or multiple phase agents of the method according to the invention.

Finally, oil components which can be used according to the invention are also dialkyl carbonates, as are described in detail in DE-A 197 101 54, to which reference is expressly made. Dioctyl carbonates, in particular, di-2-ethylhexyl carbonate, are preferred oil components for the purposes of the present invention.

The N-actylglutamic acid present in the dual or multiple phase agents of the method according to the invention is a condensation product of glutamic acid with saturated or mono- or polyunsaturated fatty acids or fatty acid mixtures, such as, for example, oleic acid, myristic acid, lauric acid, capric acid, palmitic acid, undecylenic acid, coconut fatty acid and abietic acid. These condensation products can also be in the form of salts, in particular, sodium, potassium, magnesium and triethanolamine salts.

According to the invention, it is preferred if the N-acylglutamic acid in the dual or multiple phase agents is chosen from N—(C₆-C₃₀)-acylglutamic acid and salts thereof.

Examples of commercially available N-acylglutamic acids or salts thereof according to the invention are Plantapon® ACG 35 (Cognis Deutschland), Protelan® AG 8 (Zschimmer & Schwarz Italiana), Protelan® AG 100 (Zschimmer & Schwarz Italiana), Protelan® AG 110/N (Zschimmer & Schwarz Italiana), Sepifeel One® (Seppic), Protelan® AG 89/K (Zschimmer & Schwarz Italiana), Amisoft® ECS-22 (Ajinomoto USA), Amisoft® LS-22 (Ajinomoto USA), Amisoft® HS-22 (Ajinomoto USA) and Acylglutamate® MK-11 (Ajinomoto USA).

The N-acylglutamic acid or salts thereof is present in the agents preferably in an amount of from 0.05 to 10% by weight, in particular, from 0.1 to 2% by weight, based on the weight of the agent.

The N-acylglutamic acid or salts thereof is preferably present in the agents in a weight ratio to the oil component of from 1:200 to 1:7, in particular, from 1:100 to 1:5.

According to the invention, in addition to the specified oil components, it is also possible to use alcohols which have limited miscibility with water.

Alcohols which “have limited miscibility with water” are understood as meaning those which are soluble in water at 20° C. in an amount of not more than 10% by weight, based on the mass of water.

In many cases, triols and in particular, diols have proven to be particularly suitable according to the invention. Alcohols having 4 to 20, in particular, 4 to 10, carbon atoms can be used according to the invention. The alcohols used according to the invention may be saturated or unsaturated and linear, branched or cyclic. For the purposes of the invention, it is possible to use, for example, butanol-1, cyclohexanol, pentanol-1, decanol, octanol, octenol, dodecenol, decenol, octadienol, dodecadienol, decadienol, oleyl alcohol, erucyl alcohol, ricinol alcohol, stearyl alcohol, isostearyl alcohol, cetyl alcohol, lauryl alcohol, myristyl alcohol, arachidyl alcohol, capryl alcohol, capric alcohol, linoleyl alcohol, linolenyl alcohol and behenyl alcohol, and Guerbet alcohols thereof, the intention being for this list to be exemplary and nonlimiting in character. However, the fatty alcohols originate from preferably natural fatty acids. It is usually possible to prepare them through an extraction from the esters of the fatty acids by reduction. According to the invention, it is likewise possible to use those fatty alcohol segments which are produced by reduction of naturally occurring triglycerides, such as beef tallow, palm oil, peanut oil, rapeseed oil, cottonseed oil, soy oil, sunflower oil and linseed oil or fatty acid esters arising from their transesterification products with corresponding alcohols, and thus constitute a mixture of different fatty alcohols.

Preferred alcohols according to the invention are 2-ethylhexanediol-1,3, butanol-1, cyclohexanol, pentanol-1 and 1,2-butanediol. In particular, 2-ethylhexanediol-1,3, but also butanol-1 and cyclohexanol are particularly preferred.

The invention also covers those embodiments of the method according to the invention in which the multiple phase agent is only produced directly prior to use from two or more separately formulated starting preparations. This embodiment may be preferred in cases of extremely incompatible constituents.

According to a preferred embodiment of the method according to the invention, the waving lotion is formulated in the form of the above-mentioned dual or multiple phase system. Surprisingly, it has been found that waving lotions formulated in this way produce a considerably increased waving effect for the same amount of the particular keratin-reducing components. Analogously, the waving effect achieved using a waving lotion not formulated in accordance with the invention can be achieved with a waving lotion formulated in accordance with the invention with a significant reduction in the amount of keratin-reducing substance, and thus while protecting hair and scalp.

In addition, it has been found that by formulating the waving lotion as dual and multiple phase systems, the problem of perfuming can be reduced considerably. On account of the scent note of the constituents obligatorily required in the waving lotion (keratin-reducing thio compounds, optionally alkalis, such as ammonia or alkanolamines) which are not tolerated by most users, however, perfuming is virtually essential. It is problematic that the majority of perfume components are not storage-stable in these waving lotions. The choice of scent notes for such agents is thus severely restricted. Surprisingly, it has now likewise been found that when using the dual or multiple phase systems defined according to the invention, a large number of other perfume components can be incorporated into this waving lotion in a storage-stable manner. In neutralizing solutions formulated according to the invention as well, it has now been found that additional perfume components can be incorporated in a storage-stable manner. As a result of the improved separation behavior of the agents according to the invention specified below which are used in the method according to the invention, the durability of the perfume component in dual or multiple phase agents has also been improved.

The method according to the invention is preferably used for the permanent waving or straightening of human hair.

Further subject-matters of the application are the agents used for carrying out the method according to the invention.

In the course of a method for the permanent deformation of keratin fibers, these agents serve either for carrying out the reducing stage, for carrying out the oxidizing stage or for rinsing after carrying out the reducing stage and can, in principle, comprise all constituents customary for these agents provided the conditions according to the invention (presence of the dual or multiple phase system and the short-term miscibility) are met.

Therefore, the invention secondly provides an agent for carrying out the reducing stage of a method for the permanent deformation of keratin fibers comprising a keratin-reducing substance and customary constituents, characterized in that it is in the form of a dual or multiple phase system which comprises at least one oil component and, as emulsifier, N-acylglutamic acid or salts thereof and which can be reversibly converted to a homogeneous system by mechanical movement.

The waving agents according to the invention obligatorily comprise the mercaptans known as keratin-reducing substances. Such compounds are, for example, thioglycolic acid, thiolactic acid, thiomalic acid, mercaptoethanesulfonic acid, and salts and esters thereof, cysteamine, cysteine, colored salts and salts of sulfurous acid. The alkali metal or ammonium salts of thioglycolic acid and/or of thiolactic acid, and also the free acids are preferably suitable. These are used in the waving agents preferably in concentrations of from 0.5 to 1.0 mol/kg at a pH of from 5 to 12, in particular, from 7 to 9.5. To adjust this pH, the waving agents according to the invention usually comprise alkalizing agents, such as ammonia, alkali metal and ammonium carbonates and hydrogen carbonates, or organic amines such as monoethanolamine.

In addition, the waving lotions according to the invention can comprise components which boost the waving power, such as, for example,
heterocyclic compounds, such as imidazole, pyrrolidine, piperidine, dioxolane, dioxane, morpholine and piperazine, and derivatives of these compounds, such as, for example, the C₁₋₄-alkyl derivatives, C₁₋₄-hydroxyalkyl derivatives and C₁₋₄-aminoalkyl derivatives. Preferred substituents, which may be positioned either on carbon atoms or on nitrogen atoms of the heterocyclic ring systems, are methyl, ethyl, β-hydroxyethyl and β-aminoethyl groups. Derivatives of heterocyclic compounds that are preferred according to the invention are, for example, 1-methylimidazole, 2-methylimidazole, 4(5)-methylimidazole, 1,2-dimethylimidazole, 2-ethylimidazole, 2-isopropylimidazole, N-methylpyrrolidone, 1-methylpiperidine, 4-methylpiperidine, 2-ethylpiperidine, 4-methylmorpholine, 4-(2-hydroxyethyl)morpholine, 1-ethylpiperazine, 1-(2-hydroxyethyl)piperazine, 1-(2-aminoethyl)piperazine. Furthermore, imidazole derivatives preferred according to the invention are biotin, hydantoin and benzimidazole. Very particular preference is given to imidazole;
amino acids, such as, in particular, arginine, citrulline, histidine, ornithine and lysine. The amino acids can be used either as free amino acid or as salts, e.g., as hydrochlorides. In addition, oligopeptides of on average 2-3 amino acids which have a high fraction (>50%, in particular, >70%) of the specified amino acids have also proven useful according to the invention.
Particular preference is given according to the invention to arginine and salts thereof and arginine-rich oligopeptides;
diols, such as, for example, 2-ethyl-1,3-hexanediol, 1,3-butanediol, 1,4-butanediol, 1,2-propanediol, 1,3-propanediol, neopentyl glycol and ethylene glycol. 1,3-Diols, in particular, 2-ethyl-1,3-hexanediol and 1,3-butanediol, have proven to be particularly highly suitable.

For further information regarding such components which boost the waving power, reference is made to the publications DE-A 44 36 065 and EP-B1-363 057, to the contents of which reference is expressly made.

The compounds which boost the waving power may be present in the waving lotions according to the invention in amounts of from 0.5 to 5% by weight, based on the total waving lotion. Amounts of from 1 to 4% by weight, in the case of the diols from 0.5 to 3% by weight, have proven to be sufficient, for which reason these amounts are particularly preferred.

The invention thirdly provides an agent for carrying out the oxidizing stage of a method for the permanent deformation of keratin fibers comprising an oxidizing agent and customary constituents, characterized in that it is in the form of a dual or multiple phase system which comprises at least one oil component and, as emulsifier, at least one N-acylglutamic acid or salts thereof and which can be reversibly converted to a homogeneous system by mechanical movement.

An obligatory constituent of the neutralizers according to the invention are oxidizing agents, e.g., sodium bromate, potassium bromate, hydrogen peroxide, and the stabilizers customary for stabilizing aqueous hydrogen peroxide preparations. The pH of such aqueous H₂O₂ preparations, which usually comprise about 0.5 to 15% by weight, ready-to-use usually about 0.5 to 3% by weight, of H₂O₂, is preferably 2 to 6, in particular, 2 to 4; it is adjusted using inorganic acids, preferably phosphoric acid. Neutralizers based on bromate comprise the bromates usually in concentrations of from 1 to 10% by weight and the pH of these solutions is adjusted to 4 to 7. Likewise suitable are neutralizers based on enzymes (e.g., peroxidases), which comprise no or only small amounts of oxidizing agents, in particular, H₂O₂.

The invention fourthly provides an agent for rinsing after carrying out the reducing stage of a method for the permanent deformation of keratin fibers comprising customary constituents, characterized in that it is in the form of a dual or multiple phase system which comprises at least one oil component and, as emulsifier, at least one N-acylglutamic acid or salts thereof and which can be reversibly converted into a homogeneous system by mechanical movement.

It is preferable in the above-mentioned agents according to the invention to use the oil components and N-acylglutamic acids or salts thereof and also in the embodiments specified in the first subject-matter of the invention.

According to the invention, it may be preferred, in one embodiment of the agents according to the invention, to use the N-acylglutamic acid or salts thereof as the sole emulsifier in the agents according to the invention. In addition, within the scope of this embodiment, it may be preferred to use the N-acylglutamic acid or salts thereof as the sole N-acylamino acid in the agents according to the invention.

In a further embodiment of the above-mentioned dual or multiple phase agents, it may be preferred to additionally add to the agents at least one alcohol which has only limited miscibility with water. The preferred variants of this embodiment have already been described in the first subject-matter of the invention.

In addition, it has proven advantageous if the agents according to the invention comprise a care active ingredient chosen from protein hydrolyzates and derivatives thereof.

Suitable protein hydrolyzates are, in particular, elastin, collagen, keratin, milk protein, silk protein, soy protein, almond protein, pea protein, potato protein, oat protein, corn protein and wheat protein hydrolyzates. Here, vegetable-based products may be preferred according to the invention.

Suitable derivatives are, in particular, quaternized protein hydrolyzates. Examples of this class of compound are the products available commercially under the names Lamequat®L (CTFA name: Lauryidimonium Hydroxypropylamino Hydrolyzed Animal Protein; Grünau), Croquat®WKP and Gluadin®WQ. The last-mentioned product, which is vegetable-based, may be preferred.

The protein derivatives are present in the agents according to the invention preferably in amounts of from 0.1 to 10% by weight, based on the total amount of the agent. Amounts of from 0.1 to 5% by weight are preferred.

The agents according to the invention also preferably comprise at least one conditioning active ingredient.

Suitable conditioning active ingredients are preferably cationic polymers. These are usually polymers which contain a quaternary nitrogen atom, for example, in the form of an ammonium group.

Preferred cationic polymers are, for example,

quaternized cellulose derivatives, as are commercially available under the names Celquat® and Polymer JR®. The compounds Celquat® H 100, Celquat® L 200 and Polymer JR®400 are preferred quaternized cellulose derivatives,
polysiloxanes with quaternary groups,
polymeric dimethyldiallylammonium salts and copolymers thereof with esters and amides of acrylic acid and methacrylic acid. The products commercially available under the names Merquat® 100 (poly(dimethyldiallylammonium chloride)) and Merquat® 550 (dimethyldiallylammonium chloride-acrylamide copolymer) are examples of such cationic polymers,
copolymers of vinylpyrrolidone with quaternized derivatives of dialkylaminoacrylate and -methacrylate, such as, for example, vinylpyrrolidone-dimethylaminomethacrylate copolymers quaternized with diethyl sulfate. Such compounds are commercially available under the names Gafquat®734 and Gafquat®755,
vinylpyrrolidone-vinylimidazolinium methochloride copolymers, as are supplied under the name Luviqua®,
quaternized polyvinyl alcohol
   and the polymers known under the names
Polyquaternium 2,
Polyquaternium 17,
Polyquaternium 18 and
Polyquaternium 27
   with quaternary nitrogen atoms in the polymer main chain.

Also suitable as conditioning active ingredients are amphopolymers. The generic term amphopolymers includes amphoteric polymers, i.e. polymers which contain free amino groups and free —COOH or SO₃H groups in the molecule and are capable of forming internal salts, zwitterionic polymers which contain ammonium groups and —COO⁻ or —SO₃⁻ groups in the molecule, and those polymers which contain —COOH or SO₃H groups and quaternary ammonium groups. One example of an amphopolymer which can be used according to the invention is the acrylic resin obtainable under the name Amphomer®, which is a copolymer of tert-butylaminoethyl methacrylate, N-(1,1,3,3-tetramethylbutyl)acrylamide and two or more monomers from the group acrylic acid, methacrylic acid and monoesters thereof. Likewise preferred amphopolymers are composed of unsaturated carboxylic acids (e.g., acrylic and methacrylic acid), cationically derivatized unsaturated carboxylic acids (e.g., acrylamidopropyltrimethylammonium chloride) and optionally further ionic or nonionic monomers, as given, for example, in the German laid-open specification 39 29 973 and the prior art cited therein. Terpolymers of acrylic acid, methyl acrylate and methacrylamidopropyltrimonium chloride, as are commercially available under the name Merquat®2001 N, and the commercial product Merquat® 280 are particularly preferred amphopolymers according to the invention.

The cationic or amphoteric polymers are present in the preparations according to the invention preferably in amounts of from 0.1 to 5% by weight, based on the total preparation.

Also suitable as conditioning active ingredients are silicone gums, such as, for example, the commercial product Fancorsil® LIM-1, and anionic silicones, such as, for example, the product Dow Corning® 784.

Examples of cationic surfactants which can be used as conditioning active ingredients in the agents according to the invention are, in particular, quaternary ammonium compounds. Preference is given to ammonium halides, in particular, chlorides and bromides, such as alkyltrimethylammonium chlorides, dialkyldimethylammonium chlorides and trialkylmethylammonium chlorides, e.g., cetyltrimethylammonium chloride, stearyltrimethylammonium chloride, distearyldimethylammonium chloride, lauryidimethylammonium chloride, lauryldimethylbenzylammonium chloride and tricetylmethylammoniuum chloride. It is also possible to use the very readily biodegradable quaternary ester compounds, so-called ester quats, such as, for example, the methylhydroxyalkyldialkyloxyalkylammonium methosulfates sold under the trade names Dehyquart® and Stepantex®.

Alkylamidoamines, in particular, fatty acid amidoamines, such as the stearylamidopropyldimethylamine available under the name Tego Amid®S 18, are characterized not only by a good conditioning effect, but specifically by their good biodegradability.

In addition, it may be preferred to color the individual phases with dyes in order to achieve a particularly good visual appearance of the agent. These dyes are preferably soluble only in the aqueous phase or only in at least one nonaqueous phase in an amount which allows a corresponding coloration to appear visible for the viewer. It is also possible to color both the nonaqueous phase and also the aqueous phase with different dyes, preferably in different colors. However, the mere coloring of a nonaqueous phase is preferred.

Further customary constituents for the agents according to the invention are:
anionic surfactants, such as, for example, soaps, alkyl sulfates and alkyl polyglycol ether sulfates, salts of ether carboxylic acids of the formula R—O—(CH₂—CH₂O)ₓ—CH₂—COOH, in which R is a linear alkyl group having 10 to 22 carbon atoms and x=0 or 1 to 16, acyl sarcosides, acyl taurides, acyl isethionates, sulfosuccinic acid mono- and dialkyl esters, linear alkanesulfonates, linear alpha-olefinsulfonates, alpha-sulfo fatty acid methyl esters and esters of tartaric acid and citric acid alkyl glycosides or alcohols, which constitute addition products of about 2-15 molecules of ethylene oxide and/or propylene oxide onto fatty alcohols having 8 to 22 carbon atoms,
zwitterionic surfactants, such as, for example, betaines and 2-alkyl-3-carboxymethyl-3-hydroxyethylimidazolines,
ampholytic surfactants, such as, for example, N-alkylglycines, N-alkylpropionic acids, N-alkylaminobutyric acids, N-alkyliminodipropionic acids, N-hydroxyethyl-N-alkylamidopropylglycines, N-alkyltaurines, N-alkylsarcosines, 2-alkylaminopropionic acids and alkylaminoacetic acids,
nonionic surfactants, such as, for example, addition products of from 2 to 30 mol of ethylene oxide and/or 0 to 5 mol of propylene oxide onto linear fatty alcohols having 8 to 22 carbon atoms, onto fatty acids having 12 to 22 carbon atoms and onto alkylphenols having 8 to 15 carbon atoms in the alkyl group, C₁₂-C₂₂-fatty acid mono- and diesters of addition products of from 1 to 30 mol of ethylene oxide onto glycerol, C₈-C₂₂-alkyl mono- and oligoglycosides and ethoxylated analogs thereof, and addition products of from 5 to 60 mol of ethylene oxide onto castor oil and hydrogenated castor oil,
nonionic polymers, such as, for example, vinylpyrrolidone/vinyl acetate copolymers, polyvinylpyrrolidone and vinylpyrrolidone/vinyl acetate copolymers,
anionic polymers, such as polyacrylic and polymethacrylic acids, salts thereof, copolymers thereof with acrylic acid and methacrylic acid esters and amides and derivatives thereof which are obtained by crosslinking with polyfunctional agents, polyoxycarboxylic acids, such as polyketo- and polyaldehydocarboxylic acids and salts thereof,
and polymers and copolymers of crotonic acid with esters and amides of acrylic acid and of methacrylic acid, such as vinyl acetate-crotonic acid and vinyl acetate-vinyl propionate-crotonic acid copolymers,
organic thickeners, such as agar agar, guar gum, alginates, cellulose ethers, such as methyl- and methylhydroxypropylcellulose, gelatin, pectins and/or xanthan gum. Ethoxylated fatty alcohols, in particular, those with a restricted homolog distribution, as are on the market, for example, as commercial product under the name Arlypon®F (Henkel), alkoxylated methyl glucoside esters, such as the commercial product Glucamate® DOE 120 (Amerchol), and ethoxylated propylene glycol esters, such as the commercial product Antil® 141 (Goldschmidt), may be preferred organic thickeners,
structurants, such as glucose and maleic acid,
hair-conditioning compounds, such as phospholipids, for example, soya lecithin, egg lecithin and cephalins,
perfume oils,
solubility promoters, such as ethanol, isopropanol, ethylene glycol, propylene glycol, glycerol, diethylene glycol and ethoxylated triglycerides, and fatty alcohol ethoxylates and derivatives thereof,
antidandruff active ingredients, such as climbazole, piroctone, olamine and zinc omadine,
active ingredients, such as bisabolol, allantoin, panthenol, niacinamide, tocopherol and plant extracts,
photoprotective agents,
consistency regulators, such as sugar esters, polyol esters or polyol alkyl ethers,
fats and waxes, such as spermaceti, beeswax, montan wax, paraffins, esters, glycerides and fatty alcohols,
fatty acid alkanolamides,
complexing agents, such as EDTA, NTA, β-alanine diacetic acid and phosphonic acids,
swelling and penetration substances, such as PCA, glycerol, propylene glycol monoethyl ether, carbonates, hydrogencarbonates, guanidines, ureas, and primary, secondary and tertiary phosphates,
opacifiers, such as latex or styrene/acrylamide copolymers,
pearlizing agents, such as ethylene glycol mono- and distearate or PEG-3 distearate,
direct dyes and
propellants, such as propane/butane mixtures, N₂O, dimethyl ether, CO₂ and air.

With regard to the further constituents of the agents according to the invention and their customary use amounts, reference is expressly made to the known monographs, e.g., Umbach, Kosmetik [Cosmetics], 2nd edition, Georg Thieme Verlag, Stuttgart, N.Y., 1995, and Kh. Schrader, Grundlagen und Rezepturen der Kosmetika [Fundamentals and Formulations of Cosmetics], 2nd edition, Hüthig Buch Verlag, Heidelberg, 1989.

The examples below are intended to illustrate the invention in more detail.

### EXAMPLES

Unless noted otherwise, all data are parts by weight.

Waving Agent (2 Phases).

| | 1 | | |
|---|---|---|---|
| | (according to | | |
| | the invention) | 2 | 3 |
| Aqueous phase 1 | | | |
| Ammonium thioglycolate | 16.0 | 16.0 | 16.0 |
| (71% strength in water) | | | |
| Ammonium hydrogencarbonate | 5.0 | 5.0 | 5.0 |
| Urea | 2.0 | 2.0 | 2.0 |
| Plantapon ® ACG 35 ¹ | 0.5 | — | — |
| Protelan ® VE/K ² | — | 0.6 | — |
| Merquat ®100 ³ | 0.2 | 0.2 | 0.2 |
| Turpinal ® SL ⁴ | 0.3 | 0.3 | 0.3 |
| 2-ethylhexanediol-1,3 | 0.5 | 0.5 | 0.5 |
| Ammonia | 3.0 | 3.0 | 3.0 |
| (25% strength in water) | | | |
| Water | 62.5 | 62.4 | 63.0 |
| Oil phase 2 | | | |
| Perfume oil | 1.0 | 1.0 | 1.0 |
| Wacker Belsil ® ADM 652 ⁵ | 1.0 | 1.0 | 1.0 |
| Dow Corning ® 345 Fluid ⁶ | 8.0 | 8.0 | 8.0 |

¹ Disodium N-cocoyl glutamate (31% active substance; INCI name: Sodium Cocoyl Glutamate) (Cognis Deutschland)
² Wheat protein hydrolyzate-coconut fatty acid condensate, sodium salt (26% active substance in water; INCI name: Potassium Cocoyl Hydrolyzed Wheat Protein) (Zschimmer & Schwarz)
³ Poly(dimethyldiallylammonium chloride) (40% active substance; INCI name: Polyquaternium-6) (Ondeo Nalco)
⁴ 1-Hydroxyethane-1,1-diphosphonic acid (30% active substance in water; INCI name: Etidronic Acid) (Henkel)
⁵ Polydimethylsiloxane with amino alkyl groups (INCI name: Amodimethicone) (Wacker)
⁶ Decamethylcyclopentasiloxane (INCI name: Cyclomethicone) (Dow Corning)

The waving agents 1 to 3 were each introduced into a cylinder made of glass. Each cylinder was then subjected to 5 mixing/separation cycles. Per cycle, each waving agent was shaken in the cylinder 25 times and then, for separation, left to stand for 5 minutes. After the fifth and final mixing operation, they were left to stand for 30 minutes for the separation. The mixing and separation behavior of the waving agents was evaluated.

The 2-phase waving agent 1 according to the invention could always be converted into a homogeneous mixture by 5 mixing/separation cycles and therefore has an excellent separation behavior with reformation of the starting state.

The non-inventive 2-phase waving agent 2 could be converted into a homogeneous mixture in the 5 mixing/separation cycles, but phase separation was incomplete. The starting state of the multiple phase waving agent 2 was thus not restored.

The non-inventive waving agent 3 could not be converted into a homogeneous mixture.

1. An agent for carrying out the reducing stage of a method for the permanent deformation of keratin fibers comprising a keratin-reducing substance and customary constituents, characterized in that it is in the form of a dual or multiple phase system which comprises at least one oil component and, as emulsifier, at least one N-acylglutamic acid or salts thereof and which can be reversibly converted to a homogeneous system through mechanical movement.

2. An agent for rinsing after carrying out the reducing stage of a method for the permanent deformation of keratin fibers comprising customary constituents, characterized in that it is in the form of a dual or multiple phase system which comprises at least one oil component and, as emulsifier, at least one N-acylglutamic acid or salts thereof and which can be reversibly converted to a homogeneous system through mechanical movement.

3. An agent for carrying out the oxidizing stage of a method for the permanent deformation of keratin fibers comprising an oxidizing agent and customary constituents, characterized in that it is in the form of a dual or multiple phase system which comprises at least one oil component and, as emulsifier, at least one N-acylglutamic acid or salts thereof and which can be reversibly converted to a homogeneous system through mechanical movement.

4. The agent as claimed in claim 1, characterized in that the N-acyl-glutamic acid is chosen from an N—(C₈ to C₃₀)-acylglutamic acid or salts thereof.

5. The agent as claimed in claim 1, characterized in that the oil component is chosen from vegetable oils, paraffin oils and silicones.

6. The agent as claimed in claim 1, characterized in that an alcohol which has limited miscibility with water is additionally present.

7. The agent as claimed in claim 6, characterized in that the alcohol which has limited miscibility with water has 4 to 20 carbon atoms.

8. The agent as claimed in claim 1, characterized in that a conditioning active ingredient is present.

9. A method for the permanent deformation of keratin fibers in which the fibers are treated before and/or after a mechanical deformation with an aqueous preparation of a keratin-reducing substance, after a contact time, rinsed with a first rinse, then neutralized with an aqueous preparation of an oxidizing agent and, likewise after a contact time, rinsed and optionally after-treated, characterized in that at least one of the two aqueous preparations or the first rinse is in the form of a dual or multiple phase system which comprises at least one oil component and at least one N-acylglutamic acid or salts thereof and which is reversibly converted to a homogeneous system for application to the fibers through mechanical movement.

10. The method as claimed in claim 9, characterized in that the oil component is chosen from vegetable oils, paraffin oils and silicones.

11. The method as claimed in claim 9, characterized in that the N-acyl-glutamic acid is chosen from an N—(C₈ to C₃₀)-acylglutamic acid or salts thereof.

12. The method as claimed in claim 9, characterized in that an alcohol which has limited miscibility with water is additionally present.

13. The method as claimed in claim 12, characterized in that the alcohol which has limited miscibility with water has 4 to 20 carbon atoms.
